(19) United States
(12) Patent Application Publication
(10) Pub. No.: US 2008/0069774 A1
(43) Pub. Date: Mar. 20, 2008

(54) PROTEOMIC ANTISENSE MOLECULAR SHIELD AND TARGETING

(76) Inventors: Lance Liotta, Bethesda, MD (US); David Geho, Blue Bell, PA (US); Emanuel Petricoin, Gainesville, VA (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/733,070

(22) Filed: Apr. 9, 2007

Related U.S. Application Data
(63) Continuation-in-part of application No. 11/600,901, filed on Nov. 17, 2006.
(60) Provisional application No. 60/737,383, filed on Nov. 17, 2005.

(51) Int. Cl. A61F 13/00; A61F 2/06; A61K 31/20; A61K 31/70; A61K 38/00; A61K 49/00; A61P 9/00; C07C 53/00; C07H 1/00; C07H 21/04; C07K 14/00; C07K 16/18

(52) U.S. Cl. 424/9.3; 424/422; 424/9.1; 514/2; 514/44; 514/558; 530/350; 530/387.1; 536/1.11; 536/24.5; 554/1; 623/1.11

(57) ABSTRACT

The present invention provides compositions and methods for shielding and directing agents to biological targets in cellular systems for therapeutic, prophylactic, and diagnostic uses. Vascular devices are also provided which have coated surfaces that contain proteomic antisense, as well as therapeutic and other biological agents attached thereto.

### CROSS-REFERENCE TO RELATED APPLICATIONS

This application is a continuation in parts (CIP) of U.S. patent application Ser. No. 11/600,901, filed Nov. 17, 2006 which claims the benefit of U.S. Provisional Application Ser. No. 60/737,383, filed Nov. 17, 2005, which is incorporated by reference herein.

The present invention provides compositions and methods for shielding and directing agents to biological targets in cellular systems for therapeutic, prophylactic, and diagnostic uses. Vascular devices are also provided which have coated surfaces that contain proteomic antisense, as well as therapeutic and other biological agents attached thereto.

### SUMMARY OF THE INVENTION

Heart disease remains the major cause of death in the United States. Although cardiovascular interventions have been very successful, a significant number of these interventions, such as saphenous vein harvesting for vascular bypass grafts, often lead to localized damage of endothelial surfaces. Damage of these endothelial cells results in exposure of the underlying extracellular matrix (FIG. 1). Within this matrix, exposed proteins, such as the collagens, bind platelets circulating within the blood. Platelet adhesion and activation can lead to graft failure due to thrombotic occlusion and loss of vessel patency at the site of the vascular intervention.

Current options for limiting occlusive thrombi formation include the inhibition of platelet function, such as through agents like clopidogrel. Examples of this approach were the subject of major clinical trials (the Clopidogrel for the Reduction of Events During Observation (CREDO) trial and the Percutaneous Coronary Intervention-Clopidogrel in Unstable angina to prevent Recurrent ischemic Events (PCI-CURE) trial. While these agents are quite effective in down regulating platelet activity, they do so universally. This can lead to bleeding elsewhere in the patient, as described with the glycoprotein IIb/IIIa inhibitors.

The invention relates to a novel strategy, the selective masking of exposed subendothelial matrix tissue with a protective shield that blocks platelet binding. Rather than a global effect upon platelet function within the host, this strategy provides targeted anti-thrombotic therapy directly at sites of endothelial cell damage. Introduction of the masking therapeutic could be done ex-vivo following vessel harvesting, prior to vessel implantation, for bypass procedures. The therapy could also be administered by direct injection into the vessel lumen during angioplasty procedures or stent placement.

Biocompatible Nanoparticles

The rapid growth of nanotechnologies is opening the door to new approaches for designing therapeutic agents. Already, within the field of cardiovascular research, nanoparticles have been utilized in a myriad of applications, from drug delivery to tissue engineering to the direct inhibition of angiogenesis. In general, the wide diversity of nanoparticle applications arises from remarkable range of materials and molecules that can be used to fashion nanoparticles. These include inorganic substances, such as quantum dots as well as protein-based nanoparticles, such as gelatin nanoparticles (200-300 nm).

One of the benefits of using proteins as a framework or scaffold for nanoparticle design is their inherent biocompatibility. Increasingly within the scientific community and also within the general population, there is interest in and concern about the potential toxicity of nanoparticles for biological organisms. In fact, initial studies indicate that endothelial cells secrete IL-8, a proinflammatory cytokine, following exposure to Co—SiO2-, and TiO2 nanoparticles. Accordingly, biodegradable, biocompatible nanoparticles are a means to leverage advances within the field of nanotechnology while at the same time creating substances less concerning from a toxicological viewpoint.

Limited protein domains or peptides can be linked to form higher order structures, such as nanoscale particles, using well described bioconjugate techniques. This approach enables the development of particles with surfaces that can interact with molecular specificity. The benefit of this is that the interactive potential of a nanoparticle can be rationally designed based upon the binding potential of the peptide/protein. Further, multiple protein species can be combined within the same nanoparticle, resulting in a multivalent particle in order to increase the binding potential for the extracellular matrix.

The pathophysiology of platelet-mediated thromobosis lends itself to treatment via nanoparticle-based therapeutics that specifically interfere with platelet adherence to exposed extracellular matrix (FIG. 2). During vascular interventions, the ECM-targeted therapeutic could be infused in order to rapidly protect and mask ECM exposed following endothelial cell damage from platelet adherence. For example, a vascular graft could be bathed in the nanoparticle solution prior to implantation for localized delivery of the masking therapeutic. For angioplasty, while blood flow is occluded, the masking nanoparticle solution could be infused to provide a protective coating prior to re-exposure to blood. A similar scenario could be envisioned for stent placement interventions.

As described hereinafter, Applicants of the instant invention have generated a prototypical masking system that forms the basis for the present application. In this regard, the ECM-masking properties of mature fibronectin that had undergone targeted pegylation were studied. The present invention aims to extend these findings through the assembly of a fleet of novel protein-based nanoparticles that home to sites of exposed extracellular matrix within damaged blood vessels in order to repel platelet adherence.

One embodiment of the present invention provides for the synthesis and/or design of biocompatible, non toxic nanoparticles that focus antiplatelet activity directly upon the site of vascular intervention-based vessel damage. Protein-based nanoparticles provide a means to reach this goal by providing targeted steric blockade. Preferably these protein-based nanoparticles utilize minimal protein motifs, thereby limiting unwanted additional activities of native proteins such as fibronectin.

Another embodiment of the present invention provides a proteomic antisense molecular shield comprising a targeting ligand associated with a particle, where the targeting ligand is capable of specifically binding to an extracellular component of a cellular system, and the particle is capable of masking the extracellular component from interacting with a component of the cellular system. Such an antisense proteomic shield can be utilized for a variety of applications, including those which a tissue injury has exposed otherwise hidden extracellular component materials. “Proteomic antisense” is defined as a protein-based masking system that targets a particular protein and masks its function, thereby neutralizing its activity. A proteomic antisense complex can also comprise additional elements, which provide other functions, e.g., through the bioconjugation of other functional groups for therapeutic and imaging purposes.

Additionally, the present invention relates to the development of a three-way bioconjugate nanoparticle. For example, polypeptides known to bind ECM molecules that are found within the basement membrane and/or extracellular matrix (ECM) of blood vessels can be linked through stepwise conjugation steps. One peptide that will form the basic building block for a protein based nanoparticle is a collagen binding peptide H-Cys-Gln-Asp-Ser-Glu-Thr-Arg-Thr-Phe-Tyr-OH (SEQ ID NO: 3). The peptide is capped at the carboxy (C) terminus with a lysine group during the initial peptide synthesis. Using a three arm PEG structure (purchased from Nektar Therapeutics) with maleimide functional groups appended to each arm (total size of the PEG structure=20 kDa), the amino terminal cysteines of the peptide is conjugated to the PEG structure. The reaction is performed using a large stoichiometric excess of peptide relative to PEG. Unreacted peptides are removed by dialysis or chromatography. As a negative control, a peptide with a similar number of amino acids but distinct amino acid sequence is synthesized as well. This control peptide will have amino the amino terminus with a cysteine residue and the carboxy terminus with a lysine residue in order to facilitate use of the same linkage strategy as is used with the collagen binding peptide. Nativfibronectin (obtained from Sigma) is used as a positive control.

The present invention also relates to a three-way bioconjugate, wherein the lysine moieties are biotinylated using routine strategies (Pierce Biotechnology [NHS-based linkage strategies]). This will enable streptavidin-based detection using plate bound assays similar to those described in the experimental section. More specifically, fibrillar collagen are coated onto plastic wells and unbound plastic are blocked with albumin. It is possible that steric hindrances, following biotinylation of the lysine groups, will interfere with either the collagen binding by the bioconjugate. If the conjugate does not bind to collagen, then alternative peptides may be synthesized with multiple glycines being used as spacer residues.

The instant invention not only relates to peptide-based ECMA-targeted nanoparticles but larger protein structures are also fully commensurate with the instant invention. A potential candidate is the collagen-binding fragment derived from native fibronectin (obtainable from Sigma). This fragment retains its three dimensional protein structure as well as its collagen binding properties, and it could be linked using the three-armed PEG system described above.

Six Way Bioconjugate Nanoparticle

Once the functionality of the three way bioconjugate nanoparticle is established via binding assays, two of the three-way bioconjugates may be linked to form a six-way bioconjugate. These are linked using a PEG (Nektar Therapeutics) with bifunctional reactive groups at both termini that form covalent linkages with lysines (such as NHS functionalities). This reaction enables six-way protein-based nanoparticles to be generated. The conjugates are then isolated using routine techniques such as chromatography. By having six peptides linked in this manner, the avidity of the complex for extracellular matrix is predicted to be enhanced. Once the particle is made, the amino termini are labeled with biotin using an NHS-biotin compound (obtained from Pierce Biotechnology) in order to perform plate-based binding assays with ECM molecules. Comparisons in binding between the three-way bioconjugate nanoparticle and the six-way bioconjugate nanoparticle can also be performed. As a control, a peptide sequence with a similar number of amino acids but a distinct amino acid sequence are synthesized and made into a six-way bioconjugate as well, to function as a negative control. This control peptide will have the amino terminus with a cysteine residue and the carboxy terminus with a lysine residue in order to facilitate use of the same linkage strategy as is used with the collagen binding peptide. As a positive control, native fibronectin are used as well (obtained from Sigma).

For example, vascular interventions, such as saphenous vein bypass grafting and angioplasty, while beneficial in restoring blood flow to compromised tissue, can be complicated by platelet-mediated thrombosis at the site of vascular damage. At the sites of vascular damage, endothelial cells are shed, exposing extracellular matrix (ECM), a potent binder of platelets. Platelet adhesion to a damaged blood vessel is the initial trigger for arterial thrombosis. An antisense molecular shield of the present invention can be utilized to mask the exposed ECM and block platelet binding, thereby inhibiting the subsequent cascade of deleterious events. More generally, the antisense shields can be used to treat any site of injury, damage, or event, which results in the exposure of ECM or other extracellular components. This approach can be utilized to coat and protect damaged tissues (such as blood vessels or body cavity linings), e.g., from pathologic platelet adhesion or other adverse events.

Proteomic antisense shields can also be utilized to coat biological and graft surfaces to impede adherence of pathological organisms, including bacterial, viruses, and fungi. They can also be used to modulate the migration of cells over surfaces (e.g., ECM surfaces) by blocking the receptors and/or chemoattractant signals that cells interact with, or by adding suitable chemoattractant molecules to the surface. Thus, the proteomic shields can be used to modify cell surfaces to impart essentially any desired property to it.

The particle component of the shield can be comprised of any material that is suitable for masking an extracellular component, including metallic nanoparticles (e.g., gold, copper, and combinations thereof, non-naturally-occurring polymeric materials, and biological molecules, such as polypeptide, lipids, nucleic acids, and carbohydrates. The particle can be homogenous, or it can be heterogeneous, comprising a plurality of components. One property of the particle is its ability to mask an extracellular component by interacting with another second component of the system. By the term “mask,” it is meant that the second component is prevented or blocked from associating with the extracellular component. This can be accomplished by directly covering or occupying the interaction site (e.g., a binding site for platelets) or by sterically hindering the second component from attaching to its “binding” site.

As explained in more detail below, the particle can further comprise a therapeutic and/or imaging agent.

The particle act, itself, can directly shield the target region (e.g., the binding site), or it can serve a scaffold for assembling a larger molecular shield which possesses the masking property. In addition, the particle can serve as a scaffold for attaching other agents, including therapeutic and imaging agents. For example, it can comprise capture ligands (e.g., an antibody, avidin, etc.) that enable attachment of any desired substance.

A proteomic antisense molecular shield of the present invention is preferably not internalized by a cell.

Where the cellular system is an organism or an organ, the extracellular component can be present at any desired site, including, but not limited to, e.g., the peritoneum, pericardium, pleural cavity, bladder, gastrointestinal tract, blood vessels (vascular tissue), and all organs, including the heart, pancreas, liver, ovary, and spleen. The extracellular component can underlie endothelial cells, as in vascular tissue, or it can underlie and support other epithelial cell types, such as the cells that line body cavities, including the abdominal cavity, pleural cavity, pericardium, gastrointestinal lumens, and bladder.

A targeting ligand can generally be composed of any substance that is capable of being specifically directed to a target (hence, “targeting ligand”). It is specific in the sense that it binds to the target of interest with higher affinity than other regions in the system. The ligand can also be comprised of any suitable material, including biological materials (such as peptide, lipids, nucleic acids, and carbohydrates, derivatives and hybrids thereof), as well non-naturally-occurring materials. Examples include, peptides, antibodies (hybrid, Fab, chimeric, single-chain, etc.), aptamers, etc.

A preferred target of the present invention is a component of the extracellular matrix. Examples of ECM components, include, but are not limited to, e.g., glycosaminoglycans (“GAGs”), proteoglycans (e.g., agrecan, betaglycan, decorin, perlecan, syndecan-1, etc.), collagens, elastins, fibronectins, laminins, heparin, and entactin. GAGs are unbranched polysaccharide chains that include, e.g., hyaluronan, chondroitin sulfate, dermatan sulfate, heparan sulfate, and keratan sulfate. Collagens are a family of fibrous proteins which are secreted by connective tissue cells, as well as other cell types. A typical collagen molecule is a triple-stranded helical structure in which three alpha-chains are wound together. There are about 25 different collagen alpha chains. Examples of collagen types include fibrillar (e.g., I, II, III, V); fibril-associated (IX, XII); network-forming (IV, VII); and transmembrane (XVII).

Preferred embodiments of the present invention include targeting ligands that bind to collagen in a position which is effective to block binding of platelets to collagen (e.g., type IV collagen). For example, particular targets include the collagen motif of Gly-Phe-Hyp-Gly-Glu-Arg (SEQ ID NO:1), or upstream or downstream residues which either flank [e.g., PVPGQAGAP] (SEQ ID NO:2), or which, when the polypeptide is folded, sterically hinder access to the recited motif. See, e.g., GenBank Accession No. NP_{—}001836 for an exemplary polypeptide sequence of human collagen. A targeting ligand can also comprise integrin alpha2 beta1, GPVI, or a fragment thereof, which is effective to bind to collagen and block binding of platelets thereto. These sequences can be routinely obtained in publicly available databases, e.g., GenBank (at ncbi.nlm.nih.gov). A targeting ligand can also bind to collagen:vWF complex (e.g., type VI collagen).

Another example of a proteomic antisense of the present invention is a gelatin binding fragment (GBF) of fibronectin linked to colloidal gold particle. The GBF can be used to targets the complex to exposed collagen. The collagen provides a binding site for the proteomic antisense. Not only is collagen a binding site for platelets, but proteomic antisense bound to it also provides steric blockade for other molecular platelet binding sites in the surrounding extracellular matrix. Colloidal gold, a nanometer sized particle, which has known protein binding properties, can be coated with a molecule such as albumin. The albumin coats and protects the gold from nonspecific protein association once introduced into a protein-rich environment. The amino acids on the surface of the albumin also function as a linkage site for the GBF which is the targeting moiety. Moreover, the complex, when fully formed, can be added to an antithrombotic therapeutic with known albumin association properties. After administration, the therapeutic molecules loaded onto the albumin can diffuse into the vascular site. This allows localized delivery of an additional therapy.

One or more molecular shields can be used in accordance with the present invention. For instance a plurality of antisense molecular shields can be utilized, where each comprises a different targeting ligand directed to a different extracellular component or a different region of the same extracellular component.

In addition to the use as a shield, a proteomic antisense of the present invention can also be used diagnostically, e.g., to image a region comprising ECM. For such use, the targeting ligand can comprise an imaging agent. Regions of interest include, e.g., unstable plaques in the coronary vasculature and body cavities, including those mentioned above. In particular, body cavities are difficult areas of the anatomy to adequately image using current radiological techniques. Nevertheless, these are important anatomical regions for clinical diagnoses. For example, in internal traumatic abdominal injuries, critical bleeding may occur undetected using classical imaging systems. An imaging agent associated with a proteomic antisense of the present invention can be directed to damaged vasculature facilitating the sensitive detection of such events.

Imaging can be conducted routinely using any suitable system, including common medical radiological techniques such as MRI, ultrasound, or CT.

For magnetic resonance imaging (MRI), suitable imaging agents, include contrast agents, gadolinium-containing compounds (e.g., gadopentate dimeglumine, gadoteridol, gadoterate meglumine, mangafodipor trisodium, etc.); superparamagentic iron oxides (SPIO); paramagnetic contrast agents; etc.

For ultrasound imaging, the proteomic antisense of the present invention can be incorporated into the shell of a nanoparticle. See, e.g., Wickline and Lanza, Circulation. 2003; 107; 1092-1095.

For Computed Tomography (CT; also known as “Computed Axial Tomography” or CAT), contrast agents comprising, e.g., iodine, barium, barium sulfate, or gastrografin, etc., can be utilized.

Diagnostic and imaging methods include, e.g., a method of detecting plaque in a coronary vasculature, comprising exposed regions of exposed extracellular components (e.g., which act as sites for platelet adhesion), comprising administering an effective amount of a proteomic antisense molecular shield which contains an imaging agent; and detecting the imaging agent using a compatible imaging modality, where the proteomic antisense molecular shield comprises a targeting agent capable of binding to ECM.

The present invention also provides methods of detecting a damaged tissue (e.g., a traumatic abdominal injury) comprising an exposed extracellular component, comprising: administering an effective amount of a proteomic antisense molecular shield which contains an imaging agent; and detecting the imaging agent using a compatible imaging modality, where the proteomic antisense molecular shield comprises a targeting agent capable of binding to ECM.

The particles of the present invention can also be associated with therapeutic agents, e.g., which promote wound healing or re-epithelialization at the injured sites; which prevent or reduce the proliferation of smooth muscle cells (e.g., where angioplasty has been performed); anti-neoplastic; and anti-SMC-proliferative agents. Examples of wound healing agents, include, e.g., FGF. Examples agents which block or reduce smooth muscle growth, include, e.g., anti-cytoskeletal agents, such as taxol and colchicine.

The therapeutic agent can be combined with the masking and/or imaging function to create particles with multi-modality purposes. Furthermore, the particles can be coated with a molecule (such as albumin) that is normally present in the body, and that makes it less reactive with the body's immune and surveillance systems. The molecule, itself, can also be complexed with therapeutic agents (such as aspirin) for localized delivery of antiplatelet therapy at sites of vascular injury. Albumin has many known drug binding interactions. The albumin coating can be used to carry antithrombotic drugs to sites of hemostatic instability.

Intravascular Devices

Proteomic antisense can also be used in vascular devices, such as synthetic grafts, stents, catheters, etc. Synthetic grafts used for vascular bypasses and as shunts for hemodialysis are susceptible to thrombotic occlusion, especially at sites where the graft is linked to an artery. This turbulent, high pressure environment is conducive to platelet activation and thrombosis. Proteomic antisense compounds can be used to coat the high-pressure, turbulent flow regions of the artery/graft interface. Although presently used grafts are treated with agents such as Teflon (or other materials to which polypeptides and other components do not adhere), thrombosis and occlusion remains a problem, perhaps because the surface is too slick, or nonsticky. The Teflon coating may interfere with the migration of endothelial cells over the graft. The lack of endothelial cells renders the graft susceptible to sudden clot formation if arterio-graft junction flow conditions create a sudden thrombotic environment. As an improvement, the inner surface of the graft vessel that is linked to the artery can be designed in accordance with the present invention to keep platelets from adhering, as well as to encourage endothelial cells to spread across the graft surface.

For example, the inner surface can be coated with ECM components that support endothelial cell spreading and growth. The ECM coating can be modified by the addition of agents (e.g., sugar moieties, or biologically active peptide, such as FGF) which stimulate or promote angiogenesis. For example, the sugar moiety H-2g, or 2-fucosyl lactose, has been reported to induce angiogenesis through a defined molecular mechanism, causing the release of basic fibroblast growth factor and vascular endothelial growth factor (e.g., Blood, 2005 March 15:105(6):2343). By adding the sugar to the coated surface, this finding is leveraged to create a graft surface that encourages endothelial cell migration and growth. Moreover, proteomic antisense that block platelets from binding to the ECM components (see above) can be further bound to the ECM coating. Thus, platelet adherence is discouraged, while at the same time, endothelial adherence is promoted. As a consequence, the inner surface can be populated with endothelial cells, providing a biosynthetic surface that mimics the lining of a normal blood vessel, but has the advantage that deleterious interactions with platelets are minimized.

In one embodiment of the present invention, synthetic material of a vascular device can coated with chemical groups that selectively bind to lysines or other reactive groups on proteins, such as the NHS crosslinker. The device can then be coated with collagen, fibronectin, and or other extracellular matrix components via the reactive groups. This provides an extracellular matrix-like surface that will support endothelial cell attachment. The platelet binding sites on the molecules comprising the matrix-like surface can be blocked through the addition of the proteomic antisense molecules as described above. These molecules can comprise, e.g., (1) a GBF targeting moiety (2) a polyethylene glycol moiety that links the GBF to colloidal gold coated with albumin, where (3) the albumin is derivatized via its lysine residues to bear 2-fucosyl lactose, or H-2g, or Type O blood group, sugar residues. Once coated with this array, the grafts can then be engrafted. The gold particles provide a means for monitoring via radiology tools, such as ultrasound, the presence of the proteomic antisense on the graft surface. The proteomic antisense can be reinfused over time to block platelet adherence as endothelial cells spread over the graft.

An alternative graft treatment is to coat the graft with NHS groups as above. Albumin coated with 2-fucosyl lactose, or H-2g, can be linked to PEG groups with NHS, for linkaged to albumin, and lysine at another end of the PEG. The pegylated, fucosylated albumin can then be incubated with the graft, coating the graft with active sugar groups that promote endothelialization and block platelet adherence.

The inner surface of the vascular device can be uniformly or differentially modified as needed. For instance, as discussed above, the junction between an artery and a synthetic graft can produce a high pressure, turbulent site that increases the risk of thrombosis and other deleterious events. This region of the vascular graft, proximal to the artery, can be preferentially treated with ECM components and proteomic antisense to create an endothelial-like surface whose properties have been modified to reduce platelet adherence. On the other hand, more distal regions can be left untreated, where an unmodified surface may be desirable. As another approach, the entire synthetic vascular graft surface may be treated with the protective antisense coatings. Essentially, any region of the graft vessel (or other vascular device) can be treated preferentially as desired.

For hemodialysis, a vascular access, such as a shunt or fistula, can be utilized which bridges an artery and vein, e.g., using a synthetic graft material. The proximal region where the graft is joined to the artery can be selectively modified in accordance with the present invention, while its more distal regions (including the portions proximal to the vein) can be left untreated. Alternatively, the entire synthetic graft may be modified in accordance with the present invention.

Embodiments of the present invention, include, e.g.,

### EMBODIMENT 1

A proteomic antisense molecular shield comprising:

a targeting ligand associated with a particle (where the particle can be comprised of any material, and can also act as scaffold for assembling a larger molecular shield),

wherein said targeting ligand is capable of specifically binding to an extracellular component of a cellular system, and said particle is capable of masking said extracellular component from interacting with a component of said cellular system.

### EMBODIMENT 2

A proteomic antisense molecular shield of embodiment 1, wherein said molecular shield comprises at least one nanoparticle and/or a macromolecular complex.

### EMBODIMENT 3

A proteomic antisense molecular shield of embodiment 1, wherein the targeting ligand comprises a polypeptide, lipid, carbohydrate, or nucleic acid.

### EMBODIMENT 4

A proteomic antisense molecular shield of embodiment 3, wherein said targeting ligand is an antibody.

### EMBODIMENT 6

A proteomic antisense molecular shield of embodiment 1, wherein said targeting ligand is capable of binding to a component of the extracellular matrix.

### EMBODIMENT 7

A proteomic antisense molecular shield of embodiment 1, wherein said extracellular matrix component is collagen.

### EMBODIMENT 8

A proteomic antisense molecular shield of embodiment 1, wherein said targeting ligand binds to collagen in a position which is effective to block binding of platelets to said collagen.

### EMBODIMENT 9

A proteomic antisense molecular shield of embodiment 8, wherein said targeting ligand binds to the collagen motif of Gly-Phe-Hyp-Gly-Glu-Arg (SEQ ID NO: 1).

### EMBODIMENT 10

A proteomic antisense molecular shield of embodiment 1, wherein said targeting ligand comprises integrin alpha2 beta1, GPVI, fibronectin, or a fragment thereof, which is effective to bind to collagen and block binding of platelets thereto.

### EMBODIMENT 11

A proteomic antisense molecular shield of embodiment 1, wherein said targeting ligand binds to collagen:vWF complex.

### EMBODIMENT 12

A plurality of antisense molecular shields of embodiment 1, wherein each comprises a different targeting ligand directed to a different extracellular component or a different region of the same extracellular component.

### EMBODIMENT 13

A proteomic antisense molecular shield of embodiment 1, wherein the particle comprises an imaging agent.

### EMBODIMENT 14

A method of blocking thrombus formation, comprising:

administering an effective amount of a proteomic antisense molecular shield of embodiment 1.

### EMBODIMENT 15

A method of detecting plaque in a coronary vasculature, comprising:

administering an effective amount of a proteomic antisense molecular shield of embodiment 13; and detecting said imaging agent.

### EMBODIMENT 16

A method of embodiment 15, wherein said imaging agent is a contrast agent for MRI.

### EMBODIMENT 17

A method of detecting a damaged tissue comprising an exposed extracellular component, comprising:

administering an effective amount of a proteomic antisense molecular shield of embodiment 13; and detecting said imaging agent.

### EMBODIMENT 18

A method delivering a therapeutic agent to an injured coronary vasculature, comprising:

administering an effective amount of a targeting ligand associated with a therapeutic agent to injured coronary vasculature, wherein said targeting ligand is capable of specifically binding to an extracellular component of a cellular system.

### EMBODIMENT 19

A method of embodiment 18, wherein said therapeutic agent is anti-proliferative or anti-neoplastic.

### EMBODIMENT 20

A method delivering an imaging agent to an injured coronary vasculature, comprising:

administering a targeting ligand associated with an imaging agent,

wherein said targeting ligand is capable of specifically binding to an extracellular component of a cellular system; and detecting said imaging agent.

### EMBODIMENT 21

In a method of imaging a tissue, wherein the improvement comprises,

administering a targeting ligand associated with an imaging agent,

wherein said targeting ligand is capable of specifically binding to an extracellular component of a cellular system.

### EMBODIMENT 22

A vascular device, comprising a synthetic graft having at least one anastomosis end and an inner surface coated with extracellular matrix components, and wherein the platelet binding sites within the extracellular matrix are shielded with a proteomic antisense of embodiment 1.

### EMBODIMENT 23

A vascular device of embodiment 22, wherein the inner surface of the anastomosis end is coated with extracellular matrix components and the platelet binding sites within the extracellular matrix are shielded with a proteomic antisense of embodiment 1.

### EMBODIMENT 24

A vascular device of the embodiment 23, where the inner surface of the synthetic graft distal to the anastomosis end is not coated with extracellular matrix components.

### EMBODIMENT 25

A vascular device, comprising a synthetic graft having at least one anastomosis end and an inner surface coated with a proteomic antisense of embodiment 1. Namely, the graft is coated with NHS groups as above, or some other crosslinking agent that binds amino acids is present. This is used to link albumin that has been previously derivatized, or coated, with 2-fucosyl lactose, or H-2g, or Type O blood group residues. The sugar-derivatized albumin is modified through the linkage of PEG (with heterobifunctional ends {one end has an NHS group or maleimide group, the other has lysine(s)}). The derivatized albumin conjugate can then be incubated with the graft, linking to the NHS via lysines at the end of the PEG, thereby coating the graft with active sugar groups that promote endothelialization and block platelet adherence.

### EMBODIMENT 26

A vascular device of embodiment 25, wherein the inner surface of the anastomosis end is coated with a proteomic antisense as described in embodiment 25. In dialysis shunt grafts, either the region proximal to the arterial anastomosis, or the region proximal to the venous anastomosis, or both anastomoses could be treated. In vascular bypass grafts, either the proximal anastomosis, or the distal anastomosis, or both anastomoses could be treated.

### EMBODIMENT 27

A vascular device of the embodiment 25, where the inner surface of the synthetic graft distal to the anastomosis end is not coated with proteomic antisense as described in embodiment 25.

### EMBODIMENT 28

A vascular device of the embodiment 25, where the inner surface of the synthetic graft, that is the proximal and distal anastomisis regions, are coated with proteomic antisense, while the intervening space is not.

### BRIEF DESCRIPTION OF THE DRAWINGS

Various features and attendant advantages of the present invention will be more fully appreciated as the same becomes better understood when considered in conjunction with the accompanying drawings, in which like reference characters designate the same or similar parts throughout the several views, and wherein:

FIG. 1 illustrates a proteomic antisense molecular shield of the invention;

FIGS. 2 (A)-(D) and 3 (A)-(D) illustrate ECM and vasculature, respectively, coated with such shields of the invention;

FIG. 4 (A)-(B) provide an illustration of the platelet binding site and/or collagen binding site of fibronectin;

FIG. 5 illustrates platelet binding to collagen in presence or absence of fibronectin (FN) or FN5KPEG;

FIGS. 6 (A) and (B) provide schematic illustrations of vascular grafts; and

FIG. 7 provides a schematic illustration of a smart graft.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

The invention will be explained below with reference to the following non-limiting examples.

### EXAMPLES

### Example 1

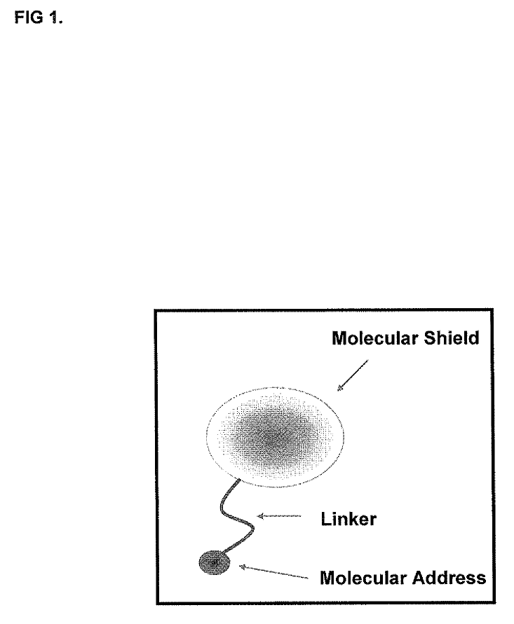

FIG. 1 shows an example of a proteomic antisense molecular shield of the present invention. A ligand to the extracellular matrix can include collagen, fibronectin, and vWF. A nanoscale molecular shield can be utilized to form a molecular blockade at the therapeutic site. The nanoparticle component can be connected to the biological molecule via a linker moiety.

### Example 2

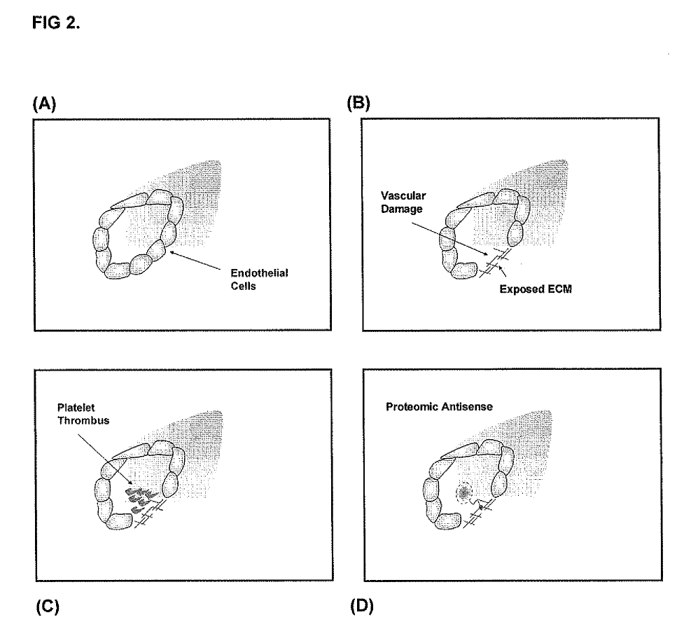

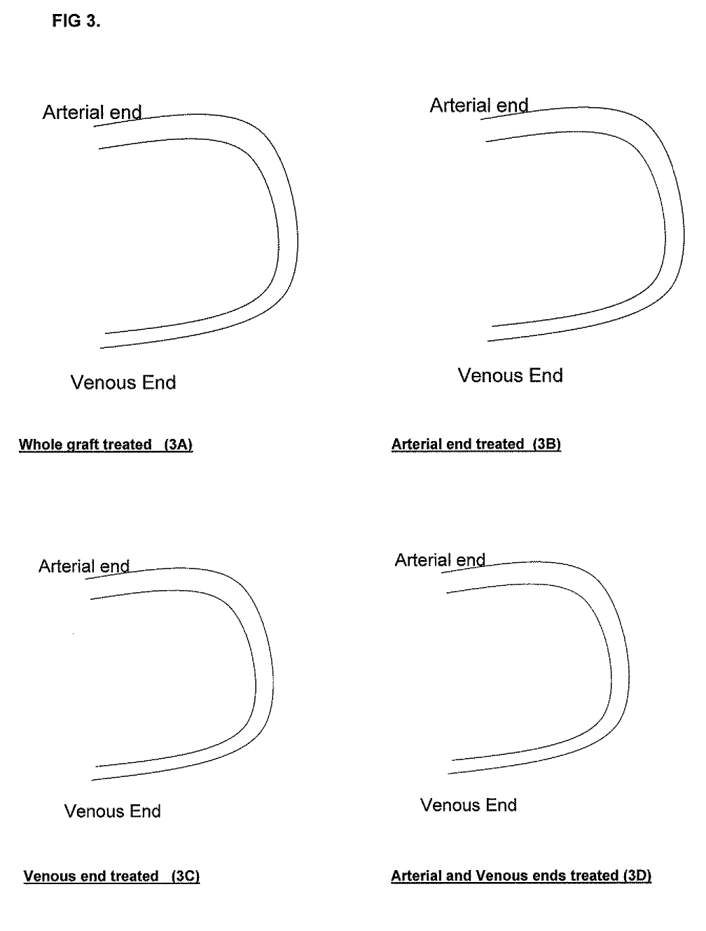

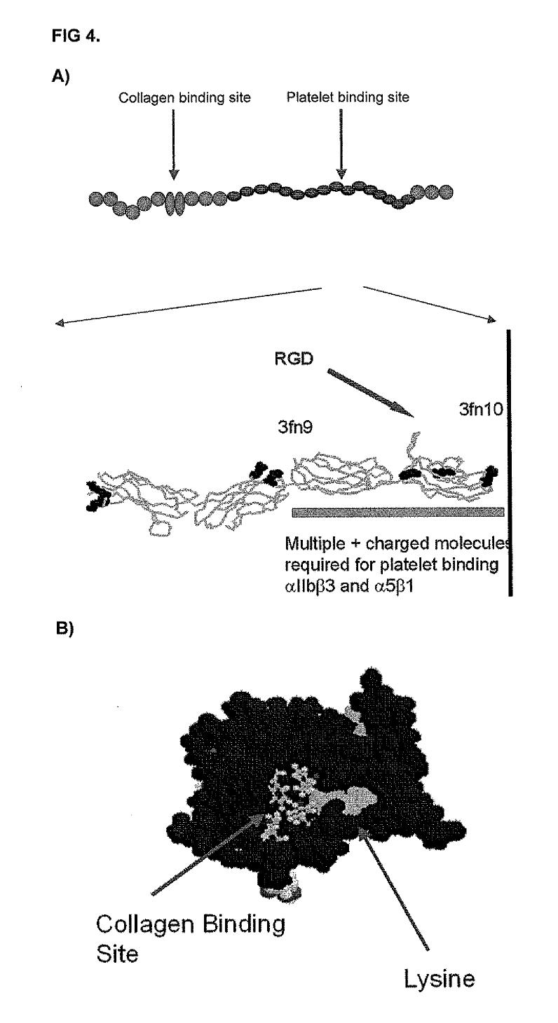

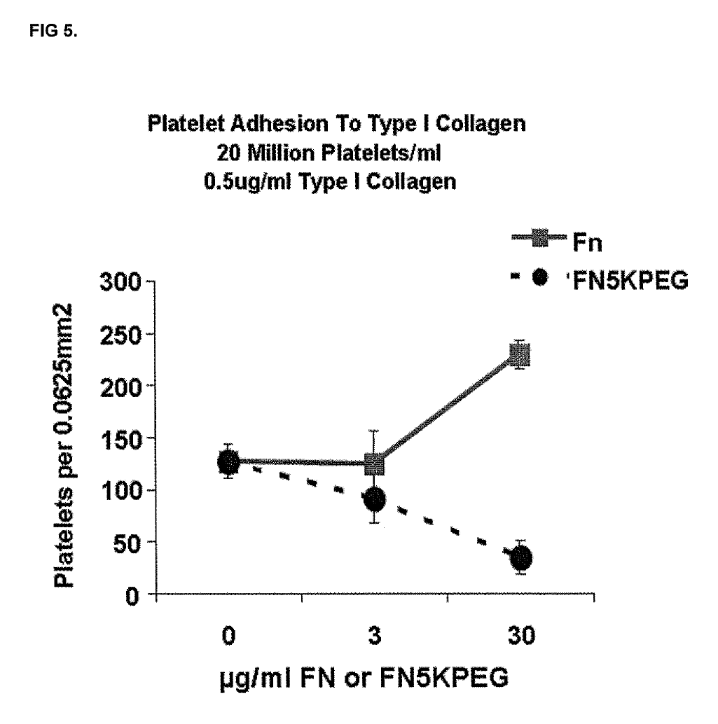

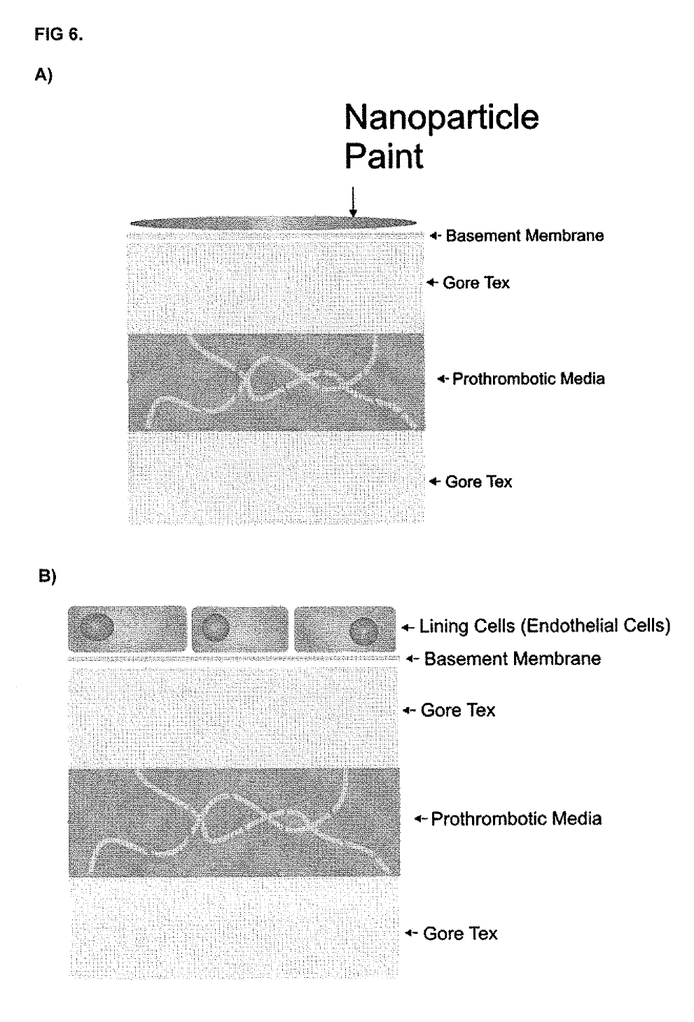

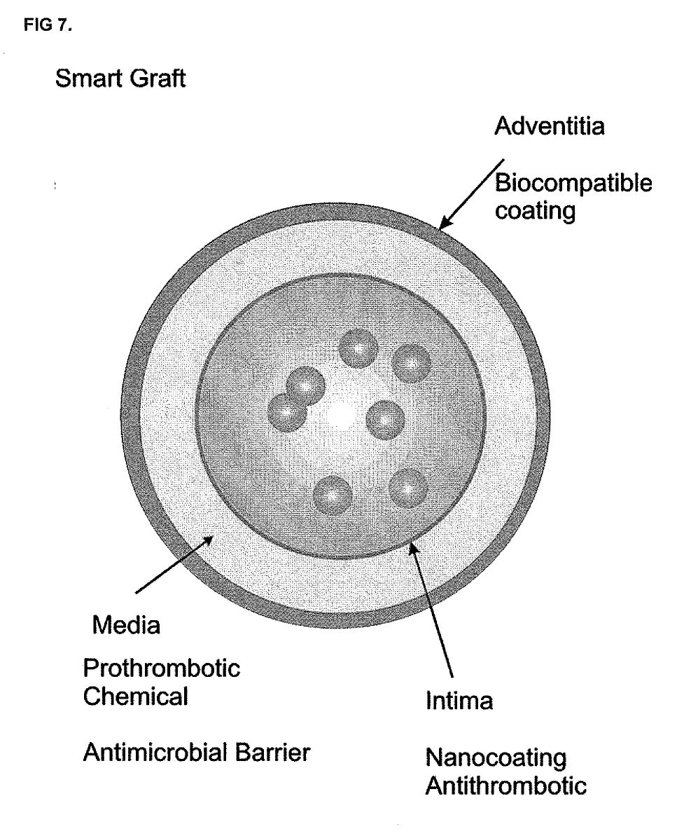

In damaged blood vessels, extracellular matrix (ECM) below the endothelial cells can be exposed. These molecules can serve as binding sites for transmembrane proteins on platelet surfaces. Binding of platelets to exposed ECM can lead to pathologic clot formation. Proteomic antisense molecular shields can be used to coat ECM, blocking unwanted platelet adhesion. See FIG. 2. Such shields can also be used to coat vascular grafts. See FIG. 3.

### Example 3

### Protein-Based Nanoparticles that Protect Vascular Intervention Sites from Platelet Adherence

Vascular interventions, such as bypass grafting, stenting, and angioplasty, have significantly altered management of patients with cardiovascular diseases. However, during these procedures the endothelial cells that form the inner lining of blood vessels can be damaged. This exposes extracellular matrix (ECM) molecules that lie underneath endothelial cells, which are potent sites for platelet binding and aggregation. For this reason, a complication of vascular interventions includes platelet activation and thrombosis. Thrombosis can seriously compromise the function of the vascular channel under repair.

Currently, treatments that target platelet function are used to decrease the likelihood of this complication. While these agents are very successful in down-regulating platelet activity, they indiscriminately attenuate the function of all circulating platelets. This can lead to bleeding elsewhere in patients.

A novel approach targets a protein-based nanoparticle therapy at the exposed ECM, which results from endothelial damage at the time of a vascular intervention, thereby blocking undesired platelet binding. This invention develops a panel of protein-based nanoparticles that bind ECM molecules, designed to block platelet adherence.

Utilizing bioconjugate techniques, protein-based molecules and biocompatible structures are used to synthesize environmentally safe, non-toxic, novel protein-based nanoparticles that are targeted to molecular constituents of ECM molecules within the wall of blood vessels. By linking ECM-binding polypeptide chains using linker molecules such as polyethylene glycol, higher order, multivalent nanoparticle structures (such as 3-way and 6-way bioconjugates) with intrinsic ECM binding properties will generated. ECM binding are documented using plate-based binding assays developed herein.

The binding capacity of protein-based nanoparticles to bind to ECM molecules within the wall of blood vessels is tested once protein-based nanoparticles are created that have demonstrated ECM-binding capabilities, binding of the nanoparticles to ECM within blood vessels harvested from patients undergoing vascular procedures are tested via immunohistochemistry. The blood vessels are damaged using a controlled system such as angioplasty balloon expansion.

The ability of the protein-based nanoparticles to block platelet adherence to ECM, including ECM in damaged blood vessels is tested with demonstration of nanoparticle adherence to ECM within damaged blood vessels, the ability of the nanoparticles to disrupt platelet binding to isolated extracellular matrix molecules in a plate-based assay are studied. Further, the nanoparticles are coated onto damaged blood vessels and the ability to disrupt platelet binding on the damaged vessel surfaces is determined.

### Example 4

As prototypical example of an ECM-targeted antiplatelet strategy, a novel bioconjugate was synthesized by this grant application's Principal Investigator. To develop a platelet blockade molecule, fibronectin (FN), an ECM and serum protein that binds platelets as well as collagen, was transformed into an ECM masking molecule. FN is a protein with multiple functions, including binding of collagen and mediating platelet adhesion. Human FN was purified from plasma using the approach of selective isolation via gelatin-agarose resin. The goal of the bioconjugation was to selectively inhibit the platelet adhesive function of FN while collagen via a targeted pegylation bioconjugate strategy.

Purified FN was then reabsorbed to gelatin agarose. Sodium cyanoborohydride and mPEG-aldehyde (MW 5,000) were added to the resin-bound FN. This reaction resulted in the covalent attachment of mPEG-aldehyde tolysine residues via reductive amination. Following this reaction and subsequent washing away of the reactants, the protein was eluted from the gelatin agarose matrix. Using this reaction sequence, the collagen binding sites within FN were not modified by the pegylation. Assessment of the proteins on SDS-PAGE showed that FNPEG-5K migrate more slowly than native FN, consistent with the fact that the modified proteins have higher molecular weights secondary to the addition of mPEG-aldehyde groups. To test whether the modified forms of FN retained the collagen-binding properties of native FN, binding studies using iodinated FN and FNPEG-5K were undertaken. These experiments indicated that FNPEG-5K bound plate-immobilized collagen in a manner similar to native FN. Thus, the key collagen-binding properties of FN were not inhibited by this targeted modification approach.

As mentioned previously, FN itself has the ability to mediate adhesion of platelets. To test whether this binding potential was eliminated following mPEG-aldehyde derivatization, FN and FNPEG-5K were immobilized onto tissue culture plates. Fluorescently labeled human mobilized proteins and adhesion was assessed. FNPEG-5K binding to platelets was markedly attenuated. This data suggests that the addition of PEG residues blocked the platelet recognition site within FN.

To further assess the ECM-binding properties FNPEG-5K, damaged blood vessels were incubated with a biotinylated form of bioconjugate. It was demonstrated that FNPEG-5K coats blood vessel wall molecules.

Since the modified FNPEG-5K retained the collagen binding properties of native FN while lacking its intrinsic platelet activating properties, the ability of this reagent to block platelet adherence to collagen was investigated. To test this, type I collagen was adsorbed to tissue culture plates. Native FN or FNPEG-5K were then incubated with the immobilized collagen and washed. Platelet adhesion was then assessed. FN-5 KPEG potently inhibited binding platelets to type I collagen, indicating that it functioned as a masking molecule for the type I collagen. Through experiments with damaged blood vessels, it was demonstrated that FNPEG-5K blocks platelet adhesion to vascular wall components.

In summary, a prototypical platelet-blocking reagent, FNPEG-5K has been derived from FN through an artificial posttranslational modification of the protein. This reagent binds to type I collagen, lacks intrinsic platelet adhesive ability, and inhibits platelet binding to a known extracellular matrix ligand.

ECM-targeted therapeutics could be applied locally at the time of vascular interventions to protect the vessels from pathologic thrombosis. It would provide platelet blockade in a defined area, unlike anti-platelet drugs that globally down-regulate platelet functions and can lead to unwanted bleeding in the host. This work was published in August 2003. See, Geho et al., Bioconjugate Chemistry. 2003 July-August; 14(4): 703-706.

These studies provided the skills and experience required to meet the objectives of the proposed research program. Bioconjugate techniques similar to the preliminary studies are used to create new protein-based nanoparticles that block platelet adherence.

The capacity of protein-based nanoparticles to bind to ECM molecules within the wall of blood vessels is tested. Once protein-based nanoparticles are created that have demonstrated ECM-binding capabilities, binding of the nanoparticles to ECM within blood vessels harvested from patients undergoing vascular procedures can be tested. More specifically, extra vein tissue from bypass grafting procedures is collected. The specimens are frozen at −70° C. for later use. These vessels are thawed at a later time and are coated with biotinylated, protein-based nanoparticles. Biotinylated control nanoparticles are used in the studies as well. The freeze-thaw cycle is sufficient to lyse the endothelial cells that line the inner surface of the blood vessel, thereby exposing the extracellular matrix molecules underneath the endothelial cells. Absence of endothelial cells is demonstrated using immunohistochemical methods (staining for endothelial cell markers such as Factor VIII or CD34). Further, the presence of type I and type IV collagen within the matrix of the vessel wall is demonstrated using immunohistochemistry. To study the ability of the nanoparticles to bind and coat damaged vasculature, the biotinylated nanoparticles are incubated with the vessels. Presence of the nanoparticles is demonstrated through probing with streptavidin linked to a reporter agent such as quantum dots. This will allow optimization of the protein-based nanoparticles for vessel binding. Sufficient blood vessels are harvested in order to experimentally optimize the study (30 vascular specimens).

In order to test the technology on fresh blood vessels (not frozen), extra vascular tissue from vascular procedures is collected and immediately placed into tissue culture media. The vessels are damaged using a controlled system. In particular, expansion of the vessels using an angioplasty balloon is used to damage the blood vessel, as described by Waksman et al. The vessel will then be infused with the protein-based nanoparticles (collagen-binding nanoparticles and control nanoparticles). As a control, non-balloon dilated vessels will also be incubated with the nanoparticles. Using multiplexed staining, endothelial cells and exposed matrix are immunostained. A similar system is used to simultaneously stain endothelial cells and matrix-bound nanoparticles. Sufficient blood vessels are harvested in order to optimize the study.

The ability of the protein-based nanoparticles to block platelet adherence to extracellular matrix, including in damaged blood vessels is tested. With demonstration of nanoparticle adherence to ECM within damaged blood vessels, the ability of the nanoparticles to disrupt platelet binding to isolated ECM molecules in a plate-based assay is studied. Using binding studies employed previously, expired platelets (after day 5) are retrieved from the Blood Bank. The platelets are labeled either with biotin or a fluorescent label. Plastic wells are coated with ECM molecules, such as type I collagen. The collagen coated wells will then be coated with the protein-based nanoparticles. Binding of platelets will then be measured. If the particles disrupt platelet binding to collagen, then the system is applied to blood vessels.

To this end, nanoparticles (collagen-targeted and control) are coated onto damaged blood vessels In order to test the technology on fresh blood vessels (not frozen). Extra vascular tissue from vascular procedures are collected and immediately placed into tissue culture media. The vessels are damaged in a controlled system using an angioplasty balloon. The damaged vessel (as well as a control vessel without balloon inflation) will then be infused with the protein-based nanoparticles (collagen-binding nanoparticles and control nanoparticles). Expired platelets labeled with either biotin or a fluorescent label are then incubated with the blood vessels. The ability of the nanoparticle preparation to disrupt platelet binding on the damaged vessel surfaces is then determined with/without flow conditions within the blood vessel. A flow device is used for fine control of platelet perfusion conditions (shear conditions) within a fresh blood vessel, as described in the literature. Along with labeled platelets, unlabeled platelets binding to blood vessels are detected using scanning electron microscopy. Sufficient blood vessels are harvested in order to optimize the study.

The entire disclosures of all applications, patents and publications, cited herein and of corresponding U.S. Provisional Application Ser. No. 60/737,383, filed Nov. 17, 2005 are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

We claim:

1. A proteomic antisense molecular shield comprising:
a targeting ligand associated with a particle,
wherein said targeting ligand is capable of specifically binding to an extracellular component of a cellular system, and said particle is capable of masking said extracellular component from interacting with a component of said cellular system.

2. A proteomic antisense molecular shield of claim 1, wherein said molecular shield comprises at least one nanoparticle and/or a macromolecular complex.

3. A proteomic antisense molecular shield of claim 1, wherein the targeting ligand comprises a polypeptide, lipid, carbohydrate, or nucleic acid.

4. A proteomic antisense molecular shield of claim 3, wherein said targeting ligand is an antibody.

5. A proteomic antisense molecular shield of claim 1, wherein said targeting ligand is capable of binding to a component of the extracellular matrix.

6. A proteomic antisense molecular shield of claim 1, wherein said extracellular matrix component is collagen.

7. A proteomic antisense molecular shield of claim 1, wherein said targeting ligand binds to collagen in a position which is effective to block binding of platelets to said collagen.

8. A proteomic antisense molecular shield of claim 8, wherein said targeting ligand binds to the collagen motif of Gly-Phe-Hyp-Gly-Glu-Arg (SEQ ID NO:1).

9. A proteomic antisense molecular shield of claim 1, wherein said targeting ligand comprises integrin alpha2 beta1, GPVI, fibronectin, or a fragment thereof which is effective to bind to collagen and block binding of platelets thereto.

10. A proteomic antisense molecular shield of claim 1, wherein said targeting ligand binds to collagen:vWF complex.

11. A plurality of antisense molecular shields of claim 1, wherein each comprises a different targeting ligand directed to a different extracellular component or a different region of the same extracellular component.

12. A proteomic antisense molecular shield of claim 1, wherein the particle comprises an imaging agent.

13. A method of blocking thrombus formation, comprising:
administering an effective amount of a proteomic antisense molecular shield of claim 1.

14. A method of detecting plaque in a coronary vasculature, comprising:
administering an effective amount of a proteomic antisense molecular shield of claim 13; and detecting said imaging agent.

15. A method of claim 15, wherein said imaging agent is a contrast agent for MRI.

16. A method of detecting a damaged tissue comprising an exposed extracellular component, comprising:
administering an effective amount of a proteomic antisense molecular shield of claim 13; and detecting said imaging agent.

17. A method delivering a therapeutic agent to an injured coronary vasculature, comprising:
administering an effective amount of a targeting ligand associated with a therapeutic agent to injured coronary vasculature, wherein said targeting ligand is capable of specifically binding to an extracellular component of a cellular system.

18. A method of claim 17, wherein said therapeutic agent is anti-proliferative or anti-neoplastic.

19. A method delivering an imaging agent to an injured coronary vasculature, comprising:
administering a targeting ligand associated with an imaging agent,
wherein said targeting ligand is capable of specifically binding to an extracellular component of a cellular system; and detecting said imaging agent.

20. In a method of imaging a tissue, wherein the improvement comprises,
administering a targeting ligand associated with an imaging agent,
wherein said targeting ligand is capable of specifically binding to an extracellular component of a cellular system.

21. A vascular device, comprising a synthetic graft having at least one anastomosis end and an inner surface coated with extracellular matrix components, and wherein the platelet binding sites within the extracellular matrix are shielded with a proteomic antisense of claim 1.

22. A vascular device of claim 21, wherein the inner surface of the anastomosis end is coated with extracellular matrix components and the platelet binding sites within the extracellular matrix are shielded with a proteomic antisense molecular shield comprising:
a targeting ligand associated with a particle which can also act as scaffold for assembling a larger molecular shield,
wherein said targeting ligand is capable of specifically binding to an extracellular component of a cellular system, and said particle is capable of masking said extracellular component from interacting with a component of said cellular system.

23. A vascular device of the claim 22, where the inner surface of the synthetic graft distal to the anastomosis end is not coated with extracellular matrix components.

24. A vascular device, comprising a synthetic graft having at least one anastomosis end and an inner surface coated with a proteomic antisense of claim 1.

25. A vascular device of claim 24, wherein the inner surface of the anastomosis end is coated with a proteomic antisense molecular shield comprising:
a targeting ligand associated with a particle which can also act as scaffold for assembling a larger molecular shield,
wherein said targeting ligand is capable of specifically binding to an extracellular component of a cellular system, and said particle is capable of masking said extracellular component from interacting with a component of said cellular system.

26. A vascular device of claim 24, where the inner surface of the synthetic graft distal to the anastomosis end is not coated with said proteomic antisense molecular shield.

13. A vascular device of claim 24, where the inner surface of the synthetic graft, comprising the proximal and distal anastomisis regions, is coated with said proteomic antisense molecular shield, while the intervening space is not.
